(19) United States
(12) Patent Application Publication
(10) Pub. No.: US 2017/0066822 A1
(43) Pub. Date: Mar. 9, 2017

(54) RESPONSIVENESS TO ANGIOGENESIS INHIBITORS

(71) Applicants: Hoffmann-La Roche Inc., Little Falls, NJ (US); VIB vzw, Gent (BE); Life Sciences Research Partners vzw, Leuven (BE)

(72) Inventors: Peter Carmeliet, Blanden (BE); Sanne Lysbet de Haas, Basel (CH); Diether Lambrechts, Kessel-lo (BE); Stefan Scherer, Preverenges (CH)

(21) Appl. No.: 15/354,454

(22) Filed: Nov. 17, 2016

Related U.S. Application Data
(63) Continuation of application No. 14/284,746, filed on May 22, 2014.
Application No. 14/284,746 is a continuation of application No. PCT/EP2012/072953, filed on Nov. 19, 2012.

(30) Foreign Application Priority Data
Nov. 23, 2011 (EP) 11190229.2

(51) Int. Cl. C07K 16/22; A61K 45/06; C12Q 1/68; A61K 39/395

(52) U.S. Cl. CPC C07K 16/22; A61K 39/3955; A61K 45/06; C12Q 1/6886; C07K 2317/24; A61K 2039/505

(57) ABSTRACT

The invention is concerned with a method of determining whether a patient is more suitably treated by a therapy with an angiogenesis inhibitor, such as bevacizumab, by determining the genotype of VEGFR-1 gene. The invention further relates to a pharmaceutical composition comprising an angiogenesis inhibitor, such as bevacizumab, for the treatment of a patient suffering from cancer based on the genotype of VEGFR-1 gene. The invention further relates to a method for improving the treatment effect of chemotherapy of a patient suffering from cancer by adding an angiogenesis inhibitor, such as bevacizumab, based on the genotype of VEGFR-1 gene.

### CROSS-REFERENCE TO RELATED APPLICATIONS

This application is a continuation of U.S. patent application Ser. No. 14/284,746, filed on May 22, 2014, which is a continuation of International Application No. PCT/EP2012/072953, having an international filing date of Nov. 19, 2012, the entire contents of which are incorporated hereby by reference, and which claims benefit under 35 U.S.C. §119 to European Patent Application No. 11190229.2, filed Nov. 23, 2011.

### SEQUENCE LISTING

The instant application contains a Sequence Listing submitted via EFS-Web and hereby incorporated by reference in its entirety. Said ASCII copy, created on Nov. 17, 2016 is named 50474-088003_Sequence_Listing_11.17.16_ST25.txt, and is 540 bytes in size.

### FIELD OF THE INVENTION

The present invention is directed to methods for identifying which patients will most benefit from treatment with anti-cancer agents and monitoring patients for their sensitivity and responsiveness to treatment with anti-cancer agents.

### BACKGROUND OF THE INVENTION

Angiogenesis contributes to benign and malignant diseases such as cancer development and, especially in cancer, is necessary for primary tumor growth, invasiveness and metastasis. In order to grow, a tumor must undergo an angiogenic switch. Vascular endothelial growth factor (VEGF) is required to induce this angiogenic switch. VEGF and the genes in the VEGF pathway are considered important mediators of cancer progression. The VEGF gene family includes the VEGF gene, also referred to as VEGFA, homologues to VEGF including, placenta growth factor (PlGF), VEGFB, VEGFC, VEGFD, the VEGF receptors, including VEGFR-1 and VEGFR-2 (also referred to as FLT1 and FLK1/KDR, respectively), the VEGF inducers, including hypoxia-inducible factors HIF1α, HIF2 α, and the oxygen sensors PHD1, PHD2 and PHD3.

The importance of this pathway in cancer cell growth and metastasis has led to the development of anti-angiogenesis agents for use in cancer therapy. These therapies include, among others, bevacizumab, pegaptanib, sunitinib, sorafenib and vatalanib. Despite significantly prolonged survival obtained with angiogenesis inhibitors, such as bevacizumab, patients still succumb to cancer. Further, not all patients respond to angiogenesis inhibitor therapy. The mechanism underlying the non-responsiveness remains unknown. Moreover, angiogenesis inhibitor therapy is associated with side effects, such as gastrointestinal perforation, thrombosis, bleeding, hypertension and proteinuria.

Accordingly, there is a need for methods of determining which patients respond particular well to angiogenesis inhibitor therapy.

It has been described in WO 2011/015348 that one or more variant alleles of the VEGFR-1 gene are associated with improved outcome of the anti-angiogenesis treatment. Among the SNPs disclosed in WO 2011/015348 are rs9554316, rs9582036, rs9513070 and rs9554320, while other SNPs are identified by linkage disequilibrium and therefore linked to these four SNPs.

### SUMMARY OF THE INVENTION

It has been found that one of the SNPs identified by linkage disequilibrium and disclosed in WO 2011/015348 is particularly useful as a predictive biomarker for the treatment outcome of an angiogenesis inhibitor, such as bevacizumab.

The present invention therefore relates to a method of determining whether a patient is more or less suitably treated by a therapy with an angiogenesis inhibitor, such as bevacizumab, by determining the genotype at the synonymous T/C SNP located in exon 28 of VEGFR-1 corresponding respectively to TAT codon and TAC codon for tyrosine at position 1213. The present invention also relates to a pharmaceutical composition comprising an angiogenesis inhibitor, such as bevacizumab, for the treatment of a patient suffering from cancer and having the genotype associated with an improved treatment effect at the synonymous T/C SNP located in exon 28 of VEGFR-1 corresponding respectively to TAT codon and TAC codon for tyrosine at position 1213. The present invention further relates to a method for improving the treatment effect of chemotherapy of a patient suffering from cancer by adding an angiogenesis inhibitor, such as bevacizumab, based on the genotype at the synonymous T/C SNP located in exon 28 of VEGFR-1 corresponding respectively to TAT codon and TAC codon for tyrosine at position 1213.

### DETAILED DESCRIPTION OF THE EMBODIMENTS

### 1. Definitions

The term “administering” means the administration of a pharmaceutical composition, such as an angiogenesis inhibitor, to the patient. For example, 2.5 mg/kg of body weight to 15 mg/kg of body weight bevacizumab (Avastin®) can be administered every week, every 2 weeks or every 3 weeks, depending on the type of cancer being treated. Particular dosages include 5 mg/kg, 7.5 mg/kg, 10 mg/kg and 15 mg/kg. Even more particular dosages are 5 mg/kg every 2 weeks, 10 mg/kg every 2 weeks and 15 mg/kg every 3 weeks.

The term “angiogenesis inhibitor” in the context of the present invention refers to all agents that alter angiogenesis (e.g. the process of forming blood vessels) and includes agents that inhibit the angiogenesis, including, but not limited to, tumor angiogenesis. In this context, inhibition can refer to blocking the formation of blood vessels and halting or slowing down the growth of blood vessels. Examples of angiogenesis inhibitors include bevacizumab (also known as Avastin®), pegaptanib, sunitinib, sorafenib and vatalanib. Bevacizumab is a recombinant humanized monoclonal IgG1 antibody that binds to and inhibits the biological activity of human VEGFA in in vitro and in vivo assay system. The term “bevacizumab” encompass all corresponding anti-VEGF antibodies that fulfill the requirements necessary for obtaining a marketing authorization as an identical or biosimilar product in a country or territory selected from the group of countries consisting of the USA, Europe and Japan. In the context of the present invention, an angiogenesis inhibitor includes an antibody that binds essentially the same epitope on VEGF as bevacizumab, more specifically an antibody that binds to the same epitope on VEGF as bevacizumab. An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in all number of different formats, using either labeled antigen or labeled antibody. Usually, the antigen is immobilized on a 96-well plate, and the ability of unlabeled antibodies to block the binding of labeled antibodies is measured using radioactive or enzyme labels.

The term “cancer” refers to the physiological condition in mammals that is typically characterized by unregulated cell proliferation. Examples of cancer include but are not limited to, carcinoma, lymphoma, blastoma, sarcoma and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer (including metastic pancreatic cancer), glioblastoma, cervical cancer, ovarian cancer, liver cancer, bladder cancer, hepatoma, breast cancer (including locally advanced, recurrent or metastatic HER-2 negative breast cancer), colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

Examples of “physiological or pathological angiogenic abnormalities” include, but are not limited to, eye disease such as age-related macular degeneration (AMD), high grade glioma, glioblastoma, M. Rendu-Osler, von-Hippel-Lindau diseases, hemangiomas, psoriasis, Kaposi's sarcoma, ocular neovascularisation, rheumatoid arthritis, endometriosis, atherosclerosis, myochardial ischemia, peripheral ischemia, cerebral ischemia and wound healing.

The term “chemotherapeutic agent” or “chemotherapy regimen” includes any active agent that can provide an anticancer therapeutic effect and may be a chemical agent or a biological agent, in particular, that are capable of interfering with cancer or tumor cells. Particular active agents are those that act as anti-neoplastic (chemotoxic or chemostatic) agents which inhibit or prevent the development, maturation or proliferation of malignant cells. Examples of chemotherapeutic agents include alkylating agents such as nitrogen mustards (e.g., mechlorethamine, cyclophosphamide, ifosfamide, melphalan and chlorambucil), nitrosoureas (e.g., carmustine (BCNU), lomustine (CCNU), and semustine (methyl-CCNU)), ethylenimines/methylmelamines (e.g., thriethylenemelamine (TEM), triethylene, thiophosphoramide (thiotepa), hexamethylmelamine (HMM, altretamine)), alkyl sulfonates (e.g., busulfan), and triazines (e.g., dacarbazine (DTIC)); antimetabolites such as folic acid analogs (e.g., methotrexate, trimetrexate), pyrimidine analogs (e.g., 5-fluorouracil, capecitabine, fluorodeoxyuridine, gemcitabine, cytosine arabinoside (AraC, cytarabine), 5-azacytidine, 2,2′-difluorodeoxycytidine), and purine analogs (e.g., 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA)); antimitotic drugs developed from natural products (e.g., paclitaxel, vinca alkaloids (e.g., vinblastine (VLB), vincristine, and vinorelbine), docetaxel, estramustine, and estramustine phosphate), epipodophylotoxins (e.g., etoposide, teniposide), antibiotics (e.g, actimomycin D, daunomycin (rubidomycin), daunorubicon, doxorubicin, epirubicin, mitoxantrone, idarubicin, bleomycins, plicamycin (mithramycin), mitomycinC, actinomycin), enzymes (e.g., L-asparaginase), and biological response modifiers (e.g., interferon-alpha, IL-2, G-CSF, GM-CSF); miscellaneous agents including platinum coordination complexes (e.g., cisplatin, carboplatin, oxaliplatin), anthracenediones (e.g., mitoxantrone), substituted urea (i.e., hydroxyurea), methylhydrazine derivatives (e.g., N-methylhydrazine (MIH), procarbazine), adrenocortical suppressants (e.g., mitotane (o,p′-DDD), aminoglutethimide); hormones and antagonists including adrenocorticosteroid antagonists (e.g, prednisone and equivalents, dexamethasone, aminoglutethimide), progestins (e.g., hydroxyprogesterone caproate, medroxyprogesterone acetate, megestrol acetate), estrogens (e.g., diethylstilbestrol, ethinyl estradiol and equivalents thereof); antiestrogens (e.g., tamoxifen), androgens (e.g., testosterone propionate, fluoxymesterone and equivalents thereof), antiandrogens (e.g., flutamide, gonadotropin-releasing hormone analogs, leuprolide), non-steroidal antiandrogens (e.g., flutamide), epidermal growth factor inhibitors (e.g., erlotinib, lapatinib, gefitinib) antibodies (e.g., trastuzumab), irinotecan and other agents such as leucovorin. For the treatment of metastatic pancreatic cancer, chemotherapeutic agents for administration with bevacizumab include gemcitabine and erlotinib and combinations thereof (see also the examples herein provided). For the treatment of renal cell cancer, chemotherapeutic agents for administration with bevacizumab include interferon alpha (see also the examples herein provided).

The term “allele” refers to a nucleotide sequence variant of a gene of interest.

The term “genotype” refers to a description of the alleles of a gene contained in an individual or a sample. In the context of this invention, no distinction is made between the genotype of an individual and the genotype of a sample originating from the individual. Although typically a genotype is determined from samples of diploid cells, a genotype can be determined from a sample of haploid cells, such as a sperm cell.

The terms “oligonucleotide” and “polynucleotide” are used interchangeably and refer to a molecule comprised of two or more deoxyribonucleotides or ribonucleotides, preferably more than three. Its exact size will depend on many factors, which in turn depend on the ultimate function or use of the oligonucleotide. An oligonucleotide can be derived synthetically or by cloning. Chimeras of deoxyribonucleotides and ribonucleotides may also be in the scope of the present invention.

The term “polymorphism” refers to the occurrence of two or more genetically determined alternative sequences of a gene in a population. Typically, the first identified allelic form is arbitrarily designated as the reference form and other allelic forms are designated as alternative or variant alleles. The allelic form occurring most frequently in a selected population is sometimes referred to as the wildtype form.

The term a “single nucleotide polymorphism” or “SNP” is a site of one nucleotide that varies between alleles. Single nucleotide polymorphisms may occur at any region of the gene. In some instances the polymorphism can result in a change in protein sequence. The change in protein sequence may affect protein function or not.

The term “patient” refers to any single animal, more specifically a mammal (including such non-human animals as, for example, dogs, cats, horses, rabbits, zoo animals, cows, pigs, sheep, and non-human primates) for which treatment is desired. Even more specifically, the patient herein is a human. In the context of the present invention, the patient may be Caucasian.

The term “subject” herein is any single human subject, including a patient, eligible for treatment who is experiencing or has experienced one or more signs, symptoms, or other indicators of an angiogenic disorder. Intended to be included as a subject are any subjects involved in clinical research trials not showing any clinical sign of disease, or subjects involved in epidemiological studies, or subjects once used as controls. The subject may have been previously treated with an anti-cancer agent, or not so treated. The subject may be naïve to an additional agent(s) being used when the treatment herein is started, i.e., the subject may not have been previously treated with, for example, an anti-neoplastic agent, a chemotherapeutic agent, a growth inhibitory agent, a cytotoxic agent at “baseline” (i.e., at a set point in time before the administration of a first dose of an anti-cancer in the treatment method herein, such as the day of screening the subject before treatment is commenced). Such “naïve” subjects are generally considered to be candidates for treatment with such additional agent(s).

The term “a patient suffering from” refers to a patient showing clinical signs in respect to a certain malignant disease, such as cancer, a disease involving physiological and pathological angiogenesis and/or tumorous disease.

As used herein, “therapy” or “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “treatment effect” encompasses the terms “overall survival” and “progression-free survival”.

The term “overall survival” refers to the length of time during and after treatment the patient survives. As the skilled person will appreciate, a patient's overall survival is improved or enhanced, if the patient belongs to a subgroup of patients that has a statistically significant longer mean survival time as compared to another subgroup of patients.

The term “progression-free survival” refers to the length of time during and after treatment during which, according to the assessment of the treating physician or investigator, the patient's disease does not become worse, i.e., does not progress. As the skilled person will appreciate, a patient's progression-free survival is improved or enhanced if the patient belongs to a subgroup of patients that has a longer length of time during which the disease does not progress as compared to the average or mean progression free survival time of a control group of similarly situated patients.

The term “pharmaceutical composition” refers to a sterile preparation that is in such form as to permit the biological activity of the medicament to be effective, and which contains no additional components that are unacceptably toxic to a subject to which the formulation would be administered.

### 2. Detailed Embodiments

In the present invention, rs7993418 SNP in the VEGFR-1 gene was identified as markers or predictive biomarkers for overall survival (OS) and/or progression-free survival (PFS) to treatment with an angiogenesis inhibitor. The terms “marker” and “predictive biomarker” can be used interchangeably and refer to specific allele variants of genes. The variation or marker may also be referred to as a single nucleotide polymorphism (SNP). Sequence information on the SNP as well as an amino acid and nucleic acid of VEGFR-1 is available on the NCBI website using respective reference/accession numbers, e.g. rs7993418, NP_002010 and NM_002019. Sequence information of rs7993418 is further shown in Table 1. In the context of the present invention, the term “VEGFR-1” also encompasses variants and/or isoforms thereof.

TABLE 1

| | | mRNA | |
|---|---|---|---|
| ID | Allele | Codon | Sequence |
| rs7993418 | A/G | TAT/TAC | AGGCTCATGAACTTGAAAGCATTTA |
| | | | C[A/G]TATCTAATGAAGAAACAGA |
| | | | AAGAAT (SEQ ID NO: 1) |

In accordance with the methods of the present invention, SNPs of VEGFR-1 were analysed using the samples derived from two Phase III trials with bevacizumab, i.e. AVITA (pancreatic cancer, see, Van Cutsem, J. Clin. Oncol. 2009 27:2231-2237) and AVOREN (renal cancer, see, Escudier et al., Lancet 2007 370:2103).

As shown in the examples, the rs7993418 SNP in VEGFR-1 was identified as the functional variant underling the association between the VEGFR-1 locus represented by four tagging SNPs, i.e. rs9554316, rs9582036, rs9513070 and rs9554320, and PFS and OS in bevacizumab-treated patients from AVITA. Further, rs7993418 correlated with PFS in bevacizumab-treated patients in AVOREN (per-allele HR=1.8, P=0.033). No effect was seen in placebo subjects (per-allele HR=0.8, P=0.49), suggesting that rs7993418 can serve as a predictive marker for favourable outcome with bevacizumab treatment.

Accordingly, the present invention provides an in vitro method of determining whether a patient suffering from cancer is suitably treated by a therapy with an angiogenesis inhibitor comprising bevacizumab or an antibody that binds essentially the same epitope on VEGF as bevacizumab, said method comprising:

(a) determining in a sample derived from a patient suffering from cancer the genotype at the synonymous T/C SNP located in exon 28 of VEGFR-1 corresponding respectively to TAT codon and TAC codon for tyrosine at position 1213, and

(b) identifying said patient as more or less suitably treated by a therapy with an angiogenesis inhibitor comprising bevacizumab or an antibody that binds essentially the same epitope on VEGF as bevacizumab based on said genotype, wherein the presence of each T allele at said SNP indicates an increased likelihood that the patient is more suitably treated, or the presence of each C allele at said SNP indicates an increased likelihood that the patient is less suitably treated. In one embodiment, the method further comprises treating the patient by the therapy with an angiogenesis inhibitor.

More specifically, the present invention provides an in vitro method of determining whether a patient is suitably treated by a therapy with an angiogenesis inhibitor comprising bevacizumab or an antibody that binds essentially the same epitope on VEGF as bevacizumab, said method comprising:

(a) determining in a sample derived from a patient suffering from cancer the genotype at the synonymous T/C SNP located in exon 28 of VEGFR-1 corresponding respectively to TAT codon and TAC codon for tyrosine at position 1213, and

(b) identifying said patient as more or less suitably treated by a therapy with an angiogenesis inhibitor comprising bevacizumab or an antibody that binds essentially the same epitope on VEGF as bevacizumab based on said genotype, wherein the presence of TT or TC genotype at said SNP indicates an increased likelihood that the patient is more suitably treated than a patient having CC genotype at said SNP, or the presence of CC genotype at said SNP indicates an increased likelihood that the patient is less suitably treated than a patient having TT or TC genotype at said SNP, or

(b′) identifying a patient as more or less suitably treated by a therapy with an angiogenesis inhibitor comprising bevacizumab or an antibody that binds essentially the same epitope on VEGF as bevacizumab based on said genotype, wherein the presence of TT genotype at said SNP indicates an increased likelihood that the patient is more suitably treated than a patient having TC or CC genotype at said SNP, or the presence of TC or CC genotype at said SNP indicates an increased likelihood that the patient is less suitably treated than a patient having TT genotype at said SNP. In one embodiment, the method further comprises treating the patient by the therapy with an angiogenesis inhibitor.

The present invention further provides a pharmaceutical composition comprising an angiogenesis inhibitor comprising bevacizumab or an antibody that binds essentially the same epitope on VEGF as bevacizumab for the treatment of a patient suffering from cancer, wherein the patient has been identified as more suitably treated with the angiogenesis inhibitor by an in vitro method comprising:

(a) determining in a sample derived from a patient suffering from cancer the genotype at the synonymous T/C SNP located in exon 28 of VEGFR-1 corresponding respectively to TAT codon and TAC codon for tyrosine at position 1213, and

(b) identifying said patient as more or less suitably treated by a therapy with an angiogenesis inhibitor comprising bevacizumab or an antibody that binds essentially the same epitope on VEGF as bevacizumab based on said genotype, wherein the presence of each T allele at said SNP indicates an increased likelihood that the patient is more suitably treated, or the presence of each C allele at said SNP indicates an increased likelihood that the patient is less suitably treated.

More specifically the present invention provides a pharmaceutical composition comprising an angiogenesis inhibitor that comprises bevacizumab or an antibody that binds essentially the same epitope on VEGF as bevacizumab, for the treatment of a patient in need thereof, wherein the patient has been identified as more suitable treated with the angiogenesis inhibitor by an in vitro method comprising:

(a) determining in a sample derived from a patient suffering from cancer the genotype at the synonymous T/C SNP located in exon 28 of VEGFR-1 corresponding respectively to TAT codon and TAC codon for tyrosine at position 1213, and

(b) identifying said patient as more or less suitably treated by a therapy with an angiogenesis inhibitor comprising bevacizumab or an antibody that binds essentially the same epitope on VEGF as bevacizumab based on said genotype, wherein the presence of TT or TC genotype at said SNP indicates an increased likelihood that the patient is more suitably treated than a patient having CC genotype at said SNP, or the presence of CC genotype at said SNP indicates an increased likelihood that the patient is less suitably treated than a patient having TT or TC genotype at said SNP, or

(b′) identifying a patient as more or less suitably treated by a therapy with an angiogenesis inhibitor comprising bevacizumab or an antibody that binds essentially the same epitope on VEGF as bevacizumab based on said genotype, wherein the presence of TT genotype at said SNP indicates an increased likelihood that the patient is more suitably treated than a patient having TC or CC genotype at said SNP, or the presence of TC or CC genotype at said SNP indicates an increased likelihood that the patient is less suitably treated than a patient having TT genotype at said SNP.

The present invention further provides a method for improving the treatment effect of a chemotherapeutic agent or chemotherapy regimen of a patient suffering from cancer by adding an angiogenesis inhibitor comprising bevacizumab or an antibody that binds essentially the same epitope on VEGF as bevacizumab, said method comprising:

(a) determining in a sample derived from a patient suffering from cancer the genotype at the synonymous T/C SNP located in exon 28 of VEGFR-1 corresponding respectively to TAT codon and TAC codon for tyrosine at position 1213;

(b) identifying said patient as more suitably treated by the addition of an angiogenesis inhibitor comprising bevacizumab or an antibody that binds essentially the same epitope on VEGF as bevacizumab based on said genotype, wherein the presence of each T allele at said SNP indicates an increased likelihood that the patient is more suitably treated; and

(c) administering said angiogenesis inhibitor in combination with a chemotherapeutic agent or chemotherapy regimen to the patient identified as more suitably treated in accordance with (b).

More specifically, the present invention provides a method for improving the treatment effect of a chemotherapeutic agent or chemotherapy regimen of a patient suffering from cancer by adding an angiogenesis inhibitor comprising bevacizumab or an antibody that binds essentially the same epitope on VEGF as bevacizumab, said method comprising:

(a) determining in a sample derived from a patient suffering from cancer the genotype at the synonymous T/C SNP located in exon 28 of VEGFR-1 corresponding respectively to TAT codon and TAC codon for tyrosine at position 1213;

(b) identifying said patient as more or less suitably treated by a therapy with an angiogenesis inhibitor comprising bevacizumab or an antibody that binds essentially the same epitope on VEGF as bevacizumab based on said genotype, wherein the presence of TT or TC genotype at said SNP indicates an increased likelihood that the patient is more suitably treated than a patient having CC genotype at said SNP, or the presence of CC genotype at said SNP indicates an increased likelihood that the patient is less suitably treated than a patient having TT or TC genotype at said SNP, or

(b′) identifying a patient as more or less suitably treated by a therapy with an angiogenesis inhibitor comprising bevacizumab or an antibody that binds essentially the same epitope on VEGF as bevacizumab based on said genotype, wherein the presence of TT genotype at said SNP indicates an increased likelihood that the patient is more suitably treated than a patient having TC or CC genotype at said SNP, or the presence of TC or CC genotype at said SNP indicates an increased likelihood that the patient is less suitably treated than a patient having TT genotype at said SNP; and

(c) administering said angiogenesis inhibitor in combination with a chemotherapeutic agent or chemotherapy regimen to a patient identified as more suitably treated in accordance with (b) or (b′).

In an embodiment, whether a patient is suitably treated by a therapy with an angiogenesis inhibitor is determined in terms of whether PFS or OS is improved, more specifically whether PFS is improved.

In an embodiment, cancer is selected from the group consisting of colorectal cancer, glioblastoma, renal cancer, ovarian cancer, breast cancer, pancreatic cancer, gastric cancer and lung cancer, more specifically the group consisting of renal cancer and pancreatic cancer.

In an embodiment, a patient can be a patient diagnosed with physiological or pathological angiogenic abnormalities.

In an embodiment, the angiogenesis inhibitor is administered as a co-treatment with a chemotherapeutic agent or chemotherapy regimen. In a further embodiment, the angiogenesis inhibitor is administered with one or more agents selected from the group consisting of taxanes such as docetaxel and paclitaxel, interferon alpha, 5-fluorouracil, leucovorin, gemcitabine, erlotinib and platinum-based chemotherapeutic agents such as carboplatin, cisplatin and oxaliplatin. More specifically, the angiogenesis inhibitor is administered as a co-treatment with a chemotherapeutic agent or chemotherapy regimen selected from the group consisting of gemcitabine-erlotinib and interferon alpha. Further, the angiogenesis inhibitor may be administered as a co-treatment with radiotherapy.

In the context of the present invention, the sample is a biological sample and may be a blood and/or tissue sample. In an embodiment, the sample is a blood sample, more specifically a peripheral blood sample. In the context of the present invention, the sample is a DNA sample. The DNA sample may be germline DNA or somatic DNA, more specifically germline DNA.

In one embodiment, the genotype is determined by means of MALDI-TOF mass spectrometry. In addition to the detailed description of the detection of SNPs below, the following reference provides guidance for MALDI-TOF mass spectrometry-based SNP genotyping, e.g. Storm et al., Methods Mol. Biol. 212:241-62, 2003.

### 3. Detection of Nucleic Acid Polymorphisms

Detection techniques for evaluating nucleic acids for the presence of a SNP involve procedures well known in the field of molecular genetics. Many, but not all, of the methods involve amplification of nucleic acids. Ample guidance for performing amplification is provided in the art. Exemplary references include manuals such as PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Current Protocols in Molecular Biology, Ausubel, 1994-1999, including supplemental updates through April 2004; Sambrook & Russell, Molecular Cloning, A Laboratory Manual (3rd Ed, 2001). General methods for detection of single nucleotide polymorphisms are disclosed in Single Nucleotide Polymorphisms: Methods and Protocols, Pui-Yan Kwok, ed., 2003, Humana Press.

Although the methods typically employ PCR steps, other amplification protocols may also be used. Suitable amplification methods include ligase chain reaction (see, e.g., Wu & Wallace, Genomics 4:560-569, 1988); strand displacement assay (see, e.g. Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86:1173-1177, 1989); and self-sustained sequence replication (3SR) (Guatelli et al., Proc. Natl. Acad. Sci. USA 87:1874-1878, 1990; WO 92/08800). Alternatively, methods that amplify the probe to detectable levels can be used, such as Qβ-replicase amplification (Kramer & Lizardi, Nature 339:401-402, 1989; Lomeli et al., Clin. Chem. 35:1826-1831, 1989). A review of known amplification methods is provided, for example, by Abramson and Myers in Current Opinion in Biotechnology 4:41-47, 1993.

Detection of the genotype, haplotype, SNP, microsatellite or other polymorphism of an individual can be performed using oligonucleotide primers and/or probes. Oligonucleotides can be prepared by any suitable method, usually chemical synthesis. Oligonucleotides can be synthesized using commercially available reagents and instruments. Alternatively, they can be purchased through commercial sources. Methods of synthesizing oligonucleotides are well known in the art (see, e.g, Narang et al., Meth. Enzymol. 68:90-99, 1979; Brown et al., Meth. Enzymol. 68:109-151, 1979; Beaucage et al., Tetrahedron Lett. 22:1859-1862, 1981; and the solid support method of U.S. Pat. No. 4,458,066). In addition, modifications to the above-described methods of synthesis may be used to desirably impact enzyme behavior with respect to the synthesized oligonucleotides. For example, incorporation of modified phosphodiester linkages (e.g., phosphorothioate, methylphosphonates, phosphoamidate, or boranophosphate) or linkages other than a phosphorous acid derivative into an oligonucleotide may be used to prevent cleavage at a selected site. In addition, the use of 2′-amino modified sugars tends to favor displacement over digestion of the oligonucleotide when hybridized to a nucleic acid that is also the template for synthesis of a new nucleic acid strand.

The genotype of an individual can be determined using many detection methods that are well known in the art. Most assays entail one of several general protocols: hybridization using allele-specific oligonucleotides, primer extension, allele-specific ligation, sequencing, or electrophoretic separation techniques, e.g., single-stranded conformational polymorphism (SSCP) and heteroduplex analysis. Exemplary assays include 5′-nuclease assays, template-directed dye-terminator incorporation, molecular beacon allele-specific oligonucleotide assays, single-base extension assays, and SNP scoring by real-time pyrophosphate sequences. Analysis of amplified sequences can be performed using various technologies such as microchips, fluorescence polarization assays, and MALDI-TOF (matrix assisted laser desorption ionization-time of flight) mass spectrometry. Two methods that can also be used are assays based on invasive cleavage with Flap nucleases and methodologies employing padlock probes.

Determination of the presence or absence of a particular allele is generally performed by analyzing a nucleic acid sample that is obtained from the individual to be analyzed. Often, the nucleic acid sample comprises genomic DNA. The genomic DNA is typically obtained from blood samples, but may also be obtained from other cells or tissues.

It is also possible to analyze RNA samples for the presence of polymorphic alleles. For example, mRNA can be used to determine the genotype of an individual at one or more polymorphic sites. In this case, the nucleic acid sample is obtained from cells in which the target nucleic acid is expressed, e.g., adipocytes. Such an analysis can be performed by first reverse-transcribing the target RNA using, for example, a viral reverse transcriptase, and then amplifying the resulting cDNA; or using a combined high-temperature reverse-transcription-polymerase chain reaction (RT-PCR), as described in U.S. Pat. Nos. 5,310,652; 5,322,770; 5,561,058; 5,641,864; and 5,693,517.

Frequently used methodologies for analysis of nucleic acid samples to detect SNPs are briefly described. However, any method known in the art can be used in the invention to detect the presence of single nucleotide substitutions.

a. Allele-Specific Hybridization

This technique, also commonly referred to as allele specific oligonucleotide hybridization (ASO) (e.g., Stoneking et al., Am. J. Hum. Genet. 48:70-382, 1991; Saiki et al., Nature 324, 163-166, 1986; EP 235,726; and WO 89/11548), relies on distinguishing between two DNA molecules differing by one base by hybridizing an oligonucleotide probe that is specific for one of the variants to an amplified product obtained from amplifying the nucleic acid sample. This method typically employs short oligonucleotides, e.g. 15-20 bases in length. The probes are designed to differentially hybridize to one variant versus another. Principles and guidance for designing such probe is available in the art, e.g. in the references cited herein. Hybridization conditions should be sufficiently stringent that there is a significant difference in hybridization intensity between alleles, and producing an essentially binary response, whereby a probe hybridizes to only one of the alleles. Some probes are designed to hybridize to a segment of target DNA such that the polymorphic site aligns with a central position (e.g., in a 15-base oligonucleotide at the 7 position; in a 16-based oligonucleotide at either the 8 or 9 position) of the probe, but this design is not required.

The amount and/or presence of an allele is determined by measuring the amount of allele-specific oligonucleotide that is hybridized to the sample. Typically, the oligonucleotide is labeled with a label such as a fluorescent label. For example, an allele-specific oligonucleotide is applied to immobilized oligonucleotides representing SNP sequences. After stringent hybridization and washing conditions, fluorescence intensity is measured for each SNP oligonucleotide.

In one embodiment, the nucleotide present at the polymorphic site is identified by hybridization under sequence-specific hybridization conditions with an oligonucleotide probe or primer exactly complementary to one of the polymorphic alleles in a region encompassing the polymorphic site. The probe or primer hybridizing sequence and sequence-specific hybridization conditions are selected such that a single mismatch at the polymorphic site destabilizes the hybridization duplex sufficiently so that it is effectively not formed. Thus, under sequence-specific hybridization conditions, stable duplexes will form only between the probe or primer and the exactly complementary allelic sequence. Thus, oligonucleotides from about 10 to about 35 nucleotides in length, usually from about 15 to about 35 nucleotides in length, which are exactly complementary to an allele sequence in a region which encompasses the polymorphic site are within the scope of the invention.

In an alternative embodiment, the nucleotide present at the polymorphic site is identified by hybridization under sufficiently stringent hybridization conditions with an oligonucleotide substantially complementary to one of the SNP alleles in a region encompassing the polymorphic site, and exactly complementary to the allele at the polymorphic site. Because mismatches which occur at non-polymorphic sites are mismatches with both allele sequences, the difference in the number of mismatches in a duplex formed with the target allele sequence and in a duplex formed with the corresponding non-target allele sequence is the same as when an oligonucleotide exactly complementary to the target allele sequence is used. In this embodiment, the hybridization conditions are relaxed sufficiently to allow the formation of stable duplexes with the target sequence, while maintaining sufficient stringency to preclude the formation of stable duplexes with non-target sequences. Under such sufficiently stringent hybridization conditions, stable duplexes will form only between the probe or primer and the target allele. Thus, oligonucleotides from about 10 to about 35 nucleotides in length, usually from about 15 to about 35 nucleotides in length, which are substantially complementary to an allele sequence in a region which encompasses the polymorphic site, and are exactly complementary to the allele sequence at the polymorphic site, are within the scope of the invention.

The use of substantially, rather than exactly, complementary oligonucleotides may be desirable in assay formats in which optimization of hybridization conditions is limited. For example, in a typical multi-target immobilized-oligonucleotide assay format, probes or primers for each target are immobilized on a single solid support. Hybridizations are carried out simultaneously by contacting the solid support with a solution containing target DNA. As all hybridizations are carried out under identical conditions, the hybridization conditions cannot be separately optimized for each probe or primer. The incorporation of mismatches into a probe or primer can be used to adjust duplex stability when the assay format precludes adjusting the hybridization conditions. The effect of a particular introduced mismatch on duplex stability is well known, and the duplex stability can be routinely both estimated and empirically determined, as described above. Suitable hybridization conditions, which depend on the exact size and sequence of the probe or primer, can be selected empirically using the guidance provided herein and well known in the art. The use of oligonucleotide probes or primers to detect single base pair differences in sequence is described in, for example, Conner et al., 1983, Proc. Natl. Acad. Sci. USA 80:278-282, and U.S. Pat. Nos. 5,468,613 and 5,604,099, each incorporated herein by reference.

The proportional change in stability between a perfectly matched and a single-base mismatched hybridization duplex depends on the length of the hybridized oligonucleotides. Duplexes formed with shorter probe sequences are destabilized proportionally more by the presence of a mismatch. Oligonucleotides between about 15 and about 35 nucleotides in length are often used for sequence-specific detection. Furthermore, because the ends of a hybridized oligonucleotide undergo continuous random dissociation and re-annealing due to thermal energy, a mismatch at either end destabilizes the hybridization duplex less than a mismatch occurring internally. For discrimination of a single base pair change in target sequence, the probe sequence is selected which hybridizes to the target sequence such that the polymorphic site occurs in the interior region of the probe.

The above criteria for selecting a probe sequence that hybridizes to a specific allele apply to the hybridizing region of the probe, i.e., that part of the probe which is involved in hybridization with the target sequence. A probe may be bound to an additional nucleic acid sequence, such as a poly-T tail used to immobilize the probe, without significantly altering the hybridization characteristics of the probe. One of skill in the art will recognize that for use in the present methods, a probe bound to an additional nucleic acid sequence which is not complementary to the target sequence and, thus, is not involved in the hybridization, is essentially equivalent to the unbound probe.

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference.

In a dot-blot format, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe.

In the reverse dot-blot (or line-blot) format, the probes are immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA. A reverse line-blot detection assay is described in the example.

An allele-specific probe that is specific for one of the polymorphism variants is often used in conjunction with the allele-specific probe for the other polymorphism variant. In some embodiments, the probes are immobilized on a solid support and the target sequence in an individual is analyzed using both probes simultaneously. Examples of nucleic acid arrays are described by WO 95/11995. The same array or a different array can be used for analysis of characterized polymorphisms. WO 95/11995 also describes subarrays that are optimized for detection of variant forms of a pre-characterized polymorphism. Such a subarray can be used in detecting the presence of the polymorphisms described herein.

b. Allele-Specific Primers

Polymorphisms are also commonly detected using allele-specific amplification or primer extension methods. These reactions typically involve use of primers that are designed to specifically target a polymorphism via a mismatch at the 3′-end of a primer. The presence of a mismatch effects the ability of a polymerase to extend a primer when the polymerase lacks error-correcting activity. For example, to detect an allele sequence using an allele-specific amplification- or extension-based method, a primer complementary to one allele of a polymorphism is designed such that the 3′-terminal nucleotide hybridizes at the polymorphic position. The presence of the particular allele can be determined by the ability of the primer to initiate extension. If the 3′-terminus is mismatched, the extension is impeded.

In some embodiments, the primer is used in conjunction with a second primer in an amplification reaction. The second primer hybridizes at a site unrelated to the polymorphic position. Amplification proceeds from the two primers leading to a detectable product signifying the particular allelic form is present. Allele-specific amplification- or extension-based methods are described in, for example, WO 93/22456; U.S. Pat. Nos. 5,137,806; 5,595,890; 5,639,611; and U.S. Pat. No. 4,851,331.

Using allele-specific amplification-based genotyping, identification of the alleles requires only detection of the presence or absence of amplified target sequences. Methods for the detection of amplified target sequences are well known in the art. For example, gel electrophoresis and probe hybridization assays described are often used to detect the presence of nucleic acids.

In an alternative probe-less method, the amplified nucleic acid is detected by monitoring the increase in the total amount of double-stranded DNA in the reaction mixture, is described, e.g. in U.S. Pat. No. 5,994,056; and European Patent Publication Nos. 487,218 and 512,334. The detection of double-stranded target DNA relies on the increased fluorescence various DNA-binding dyes, e.g., SYBR Green, exhibit when bound to double-stranded DNA.

As appreciated by one in the art, allele-specific amplification methods can be performed in reaction that employ multiple allele-specific primers to target particular alleles. Primers for such multiplex applications are generally labeled with distinguishable labels or are selected such that the amplification products produced from the alleles are distinguishable by size. Thus, for example, both alleles in a single sample can be identified using a single amplification by gel analysis of the amplification product.

As in the case of allele-specific probes, an allele-specific oligonucleotide primer may be exactly complementary to one of the polymorphic alleles in the hybridizing region or may have some mismatches at positions other than the 3′-terminus of the oligonucleotide, which mismatches occur at non-polymorphic sites in both allele sequences.

c. Detectable Probes

### i) 5′-Nuclease Assay Probes

Genotyping can also be performed using a “TaqMan®” or “5′-nuclease assay”, as described in U.S. Pat. Nos. 5,210,015; 5,487,972; and 5,804,375; and Holland et al., 1988, Proc. Natl. Acad. Sci. USA 88:7276-7280. In the TaqMan® assay, labeled detection probes that hybridize within the amplified region are added during the amplification reaction. The probes are modified so as to prevent the probes from acting as primers for DNA synthesis. The amplification is performed using a DNA polymerase having 5′- to 3′-exonuclease activity. During each synthesis step of the amplification, any probe which hybridizes to the target nucleic acid downstream from the primer being extended is degraded by the 5′- to 3′-exonuclease activity of the DNA polymerase. Thus, the synthesis of a new target strand also results in the degradation of a probe, and the accumulation of degradation product provides a measure of the synthesis of target sequences.

The hybridization probe can be an allele-specific probe that discriminates between the SNP alleles. Alternatively, the method can be performed using an allele-specific primer and a labeled probe that binds to amplified product.

Any method suitable for detecting degradation product can be used in a 5′-nuclease assay. Often, the detection probe is labeled with two fluorescent dyes, one of which is capable of quenching the fluorescence of the other dye. The dyes are attached to the probe, usually one attached to the 5′-terminus and the other is attached to an internal site, such that quenching occurs when the probe is in an unhybridized state and such that cleavage of the probe by the 5′- to 3′-exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated herein by reference, describe alternative methods for detecting the degradation of probe which occurs concomitant with amplification.

### ii) Secondary Structure Probes

Probes detectable upon a secondary structural change are also suitable for detection of a polymorphism, including SNPs. Exemplified secondary structure or stem-loop structure probes include molecular beacons or Scorpion® primer/probes. Molecular beacon probes are single-stranded oligonucleic acid probes that can form a hairpin structure in which a fluorophore and a quencher are usually placed on the opposite ends of the oligonucleotide. At either end of the probe short complementary sequences allow for the formation of an intramolecular stem, which enables the fluorophore and the quencher to come into close proximity. The loop portion of the molecular beacon is complementary to a target nucleic acid of interest. Binding of this probe to its target nucleic acid of interest forms a hybrid that forces the stem apart. This causes a conformation change that moves the fluorophore and the quencher away from each other and leads to a more intense fluorescent signal. Molecular beacon probes are, however, highly sensitive to small sequence variation in the probe target (Tyagi S. and Kramer F. R., Nature Biotechnology, Vol. 14, pages 303-308 (1996); Tyagi et al., Nature Biotechnology, Vol. 16, pages 49-53 (1998); Piatek et al., Nature Biotechnology, Vol. 16, pages 359-363 (1998); Marras S. et al., Genetic Analysis: Biomolecular Engineering, Vol. 14, pages 151-156 (1999); Tpp I. et al, BioTechniques, Vol 28, pages 732-738 (2000)). A Scorpion® primer/probe comprises a stem-loop structure probe covalently linked to a primer.

d. DNA Sequencing and Single Base Extensions

SNPs can also be detected by direct sequencing. Methods include e.g. dideoxy sequencing-based methods and other methods such as Maxam and Gilbert sequence (see, e.g. Sambrook and Russell, supra).

Other detection methods include Pyrosequencing™ of oligonucleotide-length products. Such methods often employ amplification techniques such as PCR. For example, in pyrosequencing, a sequencing primer is hybridized to a single stranded, PCR-amplified, DNA template; and incubated with the enzymes, DNA polymerase, ATP sulfurylase, luciferase and apyrase, and the substrates, adenosine 5′ phosphosulfate (APS) and luciferin. The first of four deoxynucleotide triphosphates (dNTP) is added to the reaction. DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated. Apyrase, a nucleotide degrading enzyme, continuously degrades unincorporated dNTPs and excess ATP. When degradation is complete, another dNTP is added.

Another similar method for characterizing SNPs does not require use of a complete PCR, but typically uses only the extension of a primer by a single, fluorescence-labeled dideoxyribonucleic acid molecule (ddNTP) that is complementary to the nucleotide to be investigated. The nucleotide at the polymorphic site can be identified via detection of a primer that has been extended by one base and is fluorescently labeled (e.g., Kobayashi et al, Mol. Cell. Probes, 9:175-182, 1995).

e. Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution (see, e.g. Erlich, ed., PCR Technology, Principles and Applications for DNA Amplification, W. H. Freeman and Co, New York, 1992, Chapter 7).

Distinguishing of microsatellite polymorphisms can be done using capillary electrophoresis. Capillary electrophoresis conveniently allows identification of the number of repeats in a particular microsatellite allele. The application of capillary electrophoresis to the analysis of DNA polymorphisms is well known to those in the art (see, for example, Szantai, et al, J Chromatogr A. (2005) 1079 (1-2):41-9; Bjorheim and Ekstrom, Electrophoresis (2005) 26 (13):2520-30 and Mitchelson, Mol Biotechnol. (2003) 24 (1):41-68).

f. Single-Strand Conformation Polymorphism Analysis

Alleles of target sequences can be differentiated using single-strand conformation polymorphism analysis, which identifies base differences by alteration in electrophoretic migration of single stranded PCR products, as described, e.g, in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target

SNP detection methods often employ labeled oligonucleotides. Oligonucleotides can be labeled by incorporating a label detectable by spectroscopic, photochemical, biochemical, immunochemical, or chemical means. Useful labels include fluorescent dyes, radioactive labels, e.g. 32P, electron-dense reagents, enzyme, such as peroxidase or alkaline phosphatase, biotin, or haptens and proteins for which antisera or monoclonal antibodies are available. Labeling techniques are well known in the art (see, e.g. Current Protocols in Molecular Biology, supra; Sambrook & Russell, supra).

### 4. Methods of Treatment

Dosages of with bevacizumab (Avastin®) for treatments of specific cancers, according to the EMEA, are as follows. For metastatic carcinoma of the colon or rectum (mCRC) recommended dosages are 5 mg/kg or 10 mg/kg of body weight given once every 2 weeks or 7.5 mg/kg or 15 mg/kg of body weight given once every 3 weeks, for metastatic breast cancer (mBC) recommended dosages are 10 mg/kg of body weight given once every 2 weeks or 15 mg/kg of body weight given once every 3 weeks as an intravenous infusion, and for non-small cell lung cancer (NSCLC) recommended dosages are 7.5 mg/kg or 15 mg/kg of body weight given once every 3 weeks as an intravenous infusion. Clinical benefit in NSCLC patients has been demonstrated with both 7.5 mg/kg and 15 mg/kg doses. For details refer to section 5.1 Pharmacodynamic Properties, Non-small cell lung cancer (NSCLC). For advanced and/or metastatic Renal Cell Cancer (mRCC) preferred dosages are 10 mg/kg of body weight given once every 2 weeks as an intravenous infusion (in addition to platinum-based chemotherapy for up to 6 cycles of treatment followed by bevacizumab (Avastin®) as a single agent until disease progression). For gliablastoma a particular dosage is 10 mg/kg every 2 weeks.

In the context of the present invention, the angiogenesis inhibitor may be administered in addition to or as a co-therapy or a co-treatment with one or more chemotherapeutic agents administered as part of standard chemotherapy regimen as known in the art. Examples of agents included in such standard chemotherapy regimens include 5-fluorouracil, leucovorin, irinotecan, gemcitabine, erlotinib, capecitabine, taxanes, such as docetaxel and paclitaxel, interferon alpha, vinorelbine, and platinum-based chemotherapeutic agents, such as paclitaxel, carboplatin, cisplatin and oxaliplatin. Examples of co-treatments for metastatic pancreatic cancer include gemcitabine-erlotinib plus bevacizumab at a dosage of 5 mg/kg or 10 mg/kg of body weight given once every two weeks or 7.5 mg/kg or 15 mg/kg of body weight given once every three weeks. Examples of co-treatments for renal cell cancer include interferon alpha plus bevacizumab at a dosage of or 10 mg/kg of body weight given once every two weeks. Further, a patient may be co-treated with a combination of irinotecan, 5-fluorouracil, leucovorin, also referred to as IFL, as, for example, a bolus-IFL, with a combination of oxaliplatin, leucovorin, and 5-fluorouracil, also referred to a FOLFOX4 regimen, or with a combination of capecitabine and oxaliplatin, also referred to as XELOX. Accordingly, in a further embodiment of the invention, the patient suffering from a malignant disease or a disease involving physiological and pathological angiogenesis is being treated with one or more chemotherapeutic agents such as 5-fluorouracil, leucovorin, irinotecan, gemcitabine-erlotinib, capecitabine and/or platinum-based chemotherapeutic agents, such as paclitaxel, carboplatin and oxaliplatin. Examples of co-therapy or co-treatment include 5 mg/kg bevacizumab (Avastin®) every two week with bolus-IFL or 10 mg/kg bevacizumab (Avastin®) every 2 weeks with FOLFOX4 for metastatic colorectal cancer, 15 mg/kg bevacizumab (Avastin®) every 3 weeks with caboplatis/paclitaxel for non-squamous non-small cell lung cancer, and 10 mg/kg bevacizumab (Avastin®) every 2 weeks with paclitaxel for metastatic breast cancer. Further, the angiogenesis inhibitor to be administered may be administered as a co-therapy or a co-treatment with radiotherapy.

### 5. Kit

The present invention also relates to a diagnostic composition or kit comprising any of the mentioned oligonucleotides and optionally suitable means for detection.

The kit of the invention may advantageously be used for carrying out a method of the invention and could be, inter alia, employed in a variety of applications, e.g., in the diagnostic field or as a research tool. The parts of the kit of the invention can be packages individually in vials or in combination in containers or multicontainer units. Manufacture of the kit follows preferably standard procedures which are known to the person skilled in the art. The kit or diagnostic compositions may be used for detection of the one or more variant alleles in accordance with the herein-described methods of the invention, employing, for example, amplification techniques as described herein.

Accordingly, in a further embodiment of the present invention provides a kit useful for carrying out the methods herein described, comprising oligonucleotides or polynucleotides capable of determining the genotype of one or more SNPs. The oligonucleotides or polynucleotides may comprise primers and/or probes.

The present invention is further described by reference to the following non-limited figures and examples as well as WO 2011/015348 with specific reference to Examples 1 and 2 and FIGS. 1 to 16 of WO 2011/015348.

### EXAMPLES

### Patients and Methods

### Study Design

AVITA (BO17706) and AVOREN (BO17705) were multicenter, randomized phase III trials that respectively included 607 patients with metastatic pancreatic adenocarcinoma and 649 patients with metastatic renal cell carcinoma. In AVITA, patients were randomly assigned to receive gemcitabine-erlotinib plus bevacizumab (n=306) or placebo (n=301). In AVOREN, patients were randomly assigned to receive interferon alfa-2a plus bevacizumab (n=327) or placebo (n=322). Details of these studies have been described:

AVITA: Van Cutsem et al. Phase III trial of bevacizumab in combination with gemcitabine and erlotinib in patients with metastatic pancreatic cancer. J. Clinc. Oncol. 27, 2231-7 (2009)

AVOREN: Escudier B, Pluzanska A, Koralewski P, Ravaud A, Bracarda S, Szczylik C, et al. Bevacizumab plus interferon alfa-2a for treatment of metastatic renal cell carcinoma: a randomised, double-blind phase III trial. Lancet. 2007; 370 (9605):2103-2111.

Trial protocols and genetic biomarker studies were approved by the institutional review board at each site and were conducted in accordance with the Declaration of Helsinki, current US Food and Drug Administration Good Clinical Practices, and local ethical and legal requirements. All patients included in the biomarker studies provided separate written informed consent for genetic biomarker testing. Blood samples for these analyses were collected before study treatment began.

### Single Nucleotide Polymorphism Selection

The following genes in the VEGF signaling cascade were selected: the VEGF ligand, the VEGF homologs (placenta growth factor [PlGF], VEGF-B, VEGF-C, and VEGF-D [also known as c-fos-induced growth factor or FlGF]), VEGF receptor-2 (VEGFR-2 or KDR) and VEGF receptor-1 (VEGFR-1 or FLT1), regulators of hypoxia (hypoxia-inducible factor-1α [HIF1A], HIF-2α [EPAS1], the factor inhibiting HIF-1α [FIH1], the von Hippel-Lindau tumor suppressor [VHL], the histone acetyltransferase EP300), and the oxygen sensors (prolyl hydroxylase domain-containing protein-1, -2, and -3 [EGLN-2, -1, and -3], respectively). Genomic sequences up to 5 kb upstream of the translation start site and downstream of the 3′-poly-A-adenylation site of each gene were used to select SNPs from the HapMap database (release 24/phase II). Tagging SNPs were selected using the Tagger (Pe'er I, de Bakker P I, Maller J, Yelensky R, Altshuler D, Daly M J. Evaluating and improving power in whole-genome association studies using fixed marker sets. Nat Genet 2006; 38:663-7) provided in the HAPLOVIEW software package (Barrett J C, Fry B, Maller J, Daly M J. Haploview: analysis and visualization of LD and haplotype maps. Bioinformatics 2005; 21:263-5). Only common SNPs, i.e. with minor allele frequency (f)≧0.1 and r² threshold >0.8, were considered. In total, 140 tagging SNPs were selected using these criteria. Additionally, 14 SNPs located in exonic sequences and inducing non-synonymous amino acid changes at a frequency f≧0.1 were selected from the dbSNP database, as were additional SNPs in VEGF (rs699947, rs833061, rs2010963, and rs3025039), VEGFR-1 (rs111458691) and VEGFR-2 (rs2071559), which were previously reported to affect function or expression of these genes. With future analyses in mind, 24 SNPs known to increase susceptibility to hypertension and thrombosis were also included in the design. In total, 184 SNPs were thus selected for genotyping.

### Genotyping

Peripheral blood was sampled in K2EDTA Vacutainer® tubes and germline DNA was extracted from the precipitated leukocyte cell fraction. Genotyping was carried out in a blinded manner at the Vesalius Research Center (Leuven, Belgium) with MassARRAY® iPLEX Gold (Sequenom Inc, San Diego, Calif., USA). SNPs that failed in the first genotyping round were redesigned using a different set of polymerase chain reaction primers and retested. The 27 SNPs that also failed the second design were considered failures. Overall, 157 SNPs (85.3%) were successfully genotyped with an overall success rate of 98.5% in AVITA. DNA samples from AVOREN and functional validation studies were genotyped for a limited set of SNPs including rs7993418, rs9554320, rs9582036, rs9554316, and rs9513070 using MassARRAY®.

### Statistics

Nineteen SNPs occurred at a frequency f≦0.1 in AVITA and were therefore excluded from further analysis. Hardy-Weinberg equilibrium was assessed for the remaining 138 SNPs using a standard χ² with one degree of freedom. No major violations were detected. Linkage disequilibrium (LD) strength was evaluated with r² and Lewontin's D' statistic using the Haploview software package (Broad Institute, Cambridge, Mass., USA). Associations between SNP genotypes and time to event outcomes (PFS and OS) were first evaluated using the Cox proportional hazards method according to an additive genetic model. OS analysis was performed separately for each of the 138 SNPs in the bevacizumab arm only. The significance threshold for an overall type I error rate of 0.05 was set at P<0.00036 based on the Bonferroni correction for multiple comparison in AVITA. Significant SNPs identified in this step were further analyzed considering a threshold of P<0.05 and using a Cox regression analysis: (i) in the bevacizumab arm only, while adjusting for other baseline prognostic covariates; (ii) in the placebo arm only, to assess whether observed associations were independent of treatment, and (iii) in both treatment groups, to assess genotype by treatment interaction. A stepwise model selection approach was applied to the subgroup available for genetic biomarker analysis in order to identify a set of baseline covariates affecting treatment outcome. The selected variables used as adjustment covariates were: neutrophil count, C-reactive protein, and tumor location. The association of rs7993418 was replicated in AVOREN considering a threshold of P<0.05 and using Cox regression analyses similar to AVITA.

### Results

### AVITA Study Characteristics

Blood samples from AVITA were available from 160 out of 607 patients (26.4%); 6 patients were Asian and 154 were Caucasian. Since SNP frequencies differ between ethnic groups, only DNA specimens from Caucasian patients were analyzed. The genetic biomarker subgroup was comparable with the full patient cohort with respect to age and gender distribution, smoking status, OS and PFS (Table 2). The median OS in the subgroup was 7.4 and 6.7 months in the bevacizumab and placebo arms, respectively (p=0.19), and median PFS was 5.3 and 4.1 months, respectively (p=0.078).

TABLE 2
AVITA patient demographics and clinical characteristics at baseline
Demographics and clinical characteristics are given for the full AVITA trial cohort
and for the subgroup available for genetic biomarker analysis. Bev denotes bevacizumab; CI
confidence interval, GE gemcitabine-erlotinib, mo months.

| | AVITA population | | | | Biomarker subgroup | | | |
|---|---|---|---|---|---|---|---|---|
| | GE | | Bev + GE | | GE | | Bev + GE | |
| Characteristic | (N = 301) | | (N = 306) | | (N = 77) | | (N = 77) | |
| Sex - no. (%) | | | | | | | | |
| Female | 113 | (38) | 132 | (43) | 25 | (32) | 29 | (37) |
| Male | 188 | (62) | 174 | (57) | 52 | (68) | 48 | (62) |
| Age category - no. (%) | | | | | | | | |
| <65 yr | 194 | (64) | 182 | (59) | 50 | (65) | 45 | (58) |
| ≧65 yr | 107 | (36) | 124 | (41) | 27 | (35) | 32 | (42) |
| Smoking status - no. (%) | | | | | | | | |
| Current smoker | 63 | (21) | 50 | (16) | 18 | (23) | 14 | (18) |
| Past smoker | 99 | (33) | 104 | (34) | 36 | (47) | 32 | (42) |
| Never smoker | 137 | (46) | 151 | (49) | 22 | (29) | 31 | (40) |
| Unknown | 2 | (<1) | 1 | (<1) | 1 | (1) | 0 | (0) |
| Karnofsky performance status - | | | | | | | | |
| no. (%) | | | | | | | | |
| 60 | 11 | (4) | 12 | (4) | 2 | (3) | 2 | (3) |
| 70 | 26 | (9) | 28 | (9) | 5 | (6) | 6 | (8) |
| 80 | 71 | (24) | 78 | (25) | 14 | (18) | 20 | (26) |
| 90 | 120 | (40) | 119 | (39) | 37 | (48) | 30 | (39) |
| 100 | 73 | (24) | 69 | (23) | 19 | (25) | 19 | (25) |
| Visual analogue scale score for | | | | | | | | |
| pain - no. (%) | | | | | | | | |
| <20 | 137 | (61) | 162 | (64) | 50 | (75) | 47 | (67) |
| ≧20 | 89 | (39) | 91 | (36) | 17 | (25) | 23 | (33) |
| Progression-free survival | | | | | | | | |
| Patients with event - no. (%) | 295 | (98.0) | 295 | (96.4) | 76 | (98.7) | 72 | (93.5) |
| Patients without event - no. (%) | 6 | (2.0) | 11 | (3.6) | 1 | (1.3) | 5 | (6.5) |
| Median time to event - | 3.6 | (3.4-3.7) | 4.6 | (3.8-5.4) | 4.1 | (3.5-5.3) | 5.3 | (4.0-7.0) |
| mo (95% CI) | | | | | | | | |
| Hazard ratio (95% CI) | 0.74 (0.64-0.87) | | | | 0.75 (1.03-0.54) | | | |
| Overall survival | | | | | | | | |
| Patients with event - no. (%) | 277 | (92.0) | 276 | (90.2) | 75 | (97.4) | 69 | (89.6) |
| Patients without event - no. (%) | 24 | (8.0) | 30 | (9.8) | 2 | (2.6) | 8 | (10.4) |
| Median time to event - | 6.1 | (5.5-6.8) | 7.2 | (6.6-8.0) | 6.7 | (5.3; 8.9) | 7.4 | (6.1; 9.9) |
| mo (95% CI) | | | | | | | | |

| | | |
|---|---|---|
| Hazard ratio (95% CI) | 0.89 (0.76-1.05) | 0.80 (1.12-0.58) |

The rs9582036 SNP in VEGFR-1 Correlates with Bevacizumab Treatment Outcome

Of all 138 SNPs, only the rs9582036 SNP in VEGFR-1 passed the P value threshold adjusted for multiple testing. The overall effect of this SNP on OS was significant in the bevacizumab arm (per-allele HR=2.1, P=0.00014) and consistent with an additive risk effect model (FIG. 3 of WO 2011/015348). Median OS increased from 4.8 months and 6.0 months in CC and AC carriers, respectively, to 10.3 months in AA carriers. After adjustment for neutrophil count, C-reactive protein level and tumor location, the association of rs9582036 with OS in the bevacizumab arm was slightly attenuated but still significant (HR=1.9, P=0.002). Subsequent Cox regression analysis for rs9582036 in the placebo arm did not show a statistically significant correlation between OS and SNP genotypes (FIG. 4 of WO 2011/015348). A formal test of interaction between rs9582036 and treatment (bevacizumab or placebo) was statistically significant (P=0.041), indicating that rs9582036 was a predictive marker for treatment outcome in AVITA. Cox regression analysis also revealed a correlation between rs9582036 and PFS in the bevacizumab arm (per-allele HR=1.89, P=0.00081; FIG. 5 of WO 2011/015348). No such effect for PFS was observed in the placebo arm (P=0.58; FIG. 6 of WO 2011/015348).

### Associated SNPs Define a Locus in the VEGFR-1 TK Domain

Three other SNPs in VEGFR-1 (rs9554316, rs9513070, and rs9554320) also correlated with OS in the bevacizumab arm, but did not pass the P value threshold adjusted for multiple testing (P=0.00042, P=0.0081, and P=0.0097, respectively). The predictive effects of these SNPs were similar to those of rs9582036 (FIGS. 7 to 10 of WO 2011/015348). All four SNPs were located close to each other, i.e. in introns 25, 27, 28, and 29 for rs9554320, rs9582036, rs9554316, and rs9513070, respectively, and represented four consecutive regions of high linkage disequilibrium within VEGFR-1. When considering the P value of every SNP as a measure of its association with OS and plotting these values as a function of the location of the SNP in VEGFR-1, an association signal encompassing exons 25 to 29, which code for amino acid residues 1029 to 1272 in the TK domain, was observed in the bevacizumab arm. As expected, no such signal was observed in the placebo group.

### Fine-Mapping of the VEGFR-1 Locus

To identify all SNPs located in VEGFR-1, we used whole-genome sequencing data from 60 Caucasian HapMap samples in the 1000 Genomes project (CEU population, Release July 2010; www.1000genomes.org). Using the VCF Tools version 0.1.5, SNPs in the coding region of VEGFR-1 and in the 15 kb up- and downstream sequence (i.e., the Chr13:27763000-27982000 Ensembl 36.3 coordinates) were selected. In total, we identified 628 SNPs, of which 381 had a minor allele frequency (MAF) ≧0.05. Using the Haploview 4.2 software, we identified 48 SNPs that were in LD with one of the four tagging SNPs associated with treatment outcome after bevacizumab in AVITA (i.e., rs9582036, rs9554316, rs9513070 and rs9554320). The threshold for LD was set at r²≧0.12 as this was the lowest r² between one of the four tag SNPs in the samples analyzed (Table 3).

TABLE 3

| r² value | rs9513070 | rs9554316 | rs9582036 | rs9554320 |
|---|---|---|---|---|
| rs9513070 | — | 0.28 | 0.21 | 0.12 |
| rs9554316 | 0.28 | — | 0.67 | 0.33 |
| rs9582036 | 0.21 | 0.67 | — | 0.48 |
| rs9554320 | 0.12 | 0.33 | 0.48 | — |

The pairwise linkage disequilibrium between the 4 tagging SNPs in the VEGFR-1 locus is shown. SNPs, which are in perfect correlation and are completely synonymous, have a r² value of 1. SNPs with an r² value of 0 occur independently from each other.

To identify which of these 48 SNPs affect VEGFR-1 function and causally contribute to treatment outcome after bevacizumab, we used the PupaSuite (Reumers J, Conde L, Medina I, et al. Joint annotation of coding and non-coding single nucleotide polymorphisms and mutations in the SNPeffect and PupaSuite databases. Nucleic Acids Res 2008; 36:D825-9) and AnnoVar (Wang K, Li M, Hakonarson H. ANNOVAR: functional annotation of genetic variants from high-throughput sequencing data. Nucleic Acids Res; 38:e164) tools. In particular, we assessed which of these SNPs were located in coding regions, transcription factor binding sites, exonic splicing enhancers/silencers or miRNA binding sites, or in other evolutionary conserved sequence regions. Only one SNP was located in one of the VEGFR-1 exons, i.e., rs7993418 was located in exon 28 of VEGFR-1. Two SNPs (i.e., rs9513071 and rs7982283) were located in a predicted CCCTC-binding factor (CTCF) binding motif, but were unlikely to functionally affect VEGFR-1 function since they did not disrupt the core-binding domain of the CTCF motif. Five other SNPs were located on conserved positions, which were defined as conservation of the respective nucleotide position in at least 10 mammals out of the 44 species in the database. These SNPs were located downstream of the VEGFR-1 gene (rs9554309), in intronic sequences (rs9513073, rs9551471, rs7992940) and in exon 28 of VEGFR-1 (rs7993418). No other relevant SNPs were identified. Notably, of these 5 SNPs, rs7993418 showed the highest degree of LD with the four tag SNPs in the VEGFR-1 TK locus (r² values of 0.34, 0.83, 0.67 and 0.36 for LD with rs9513070, rs9554316, rs9582036 and rs9554320, respectively). Overall, based on this fine-mapping and in silico analysis, rs7993418 was considered to be the SNP with the highest potential to affect VEGFR-1 function. Rs7993418 is a synonymous T/C SNP located in exon 28 of VEGFR-1 that changes the TAT codon of tyrosine 1213 into a TAC codon (Tyr1213Tyr) and is located in the haplotype block of rs9554316.

The rs7993418 Variant Functionally Affects VEGFR-1 Expression
1. In Vitro Transcription/Translation of VEGFR-1 cDNA Constructs

To demonstrate that rs7993418 functionally affects VEGFR-1 expression, its effect on transcription and translation of VEGFR-1 cDNA was assessed in vitro using the rabbit reticulocyte lysate system. Two versions of the VEGFR-1 cDNA, carrying either the TAT or TAC codon for Tyr1213, were generated. Both cDNAs were cloned into the pcDNA3 expression vector and were used for in vitro transcription/translation using the commercial TnT T7 Quick-coupled rabbit reticulocyte lysate kit (Promega, Cat# L1170). Full-length VEGFR-1 cDNA carrying either the wild-type TAT or mutant TAC codon yielded equal amounts of transcribed mRNA but different amounts of translated VEGFR-1 protein. In particular, a 27% increase in VEGFR-1 protein was observed for TAC versus TAT-carrying cDNA constructs (P<0.001). Likewise, transient overexpression in HEK293T cells confirmed that, although VEGFR-1 mRNA expression was equal between cells expressing the TAC and TAT-carrying construct, up to 15% more VEGFR-1 protein was translated by TAC-expressing cells (P<0.001). Expression of the soluble VEGFR-1 isoform (sVEGFR-1) produced by proteolytic cleavage of full-length transmembrane VEGFR-1 (tmVEGFR-1) was similarly increased in cells expressing the TAC-carrying construct (P<0.001).

2. sVEGFR-1 Expression Levels in Human Plasma

Furthermore, since tmVEGFR-1 and sVEGFR-1 protein levels are strongly correlated and sVEGFR-1 can easily be assessed in human plasma, sVEGFR-1 plasma levels were measured in two independent cohorts and stratified for rs7993418. Plasma was collected from 369 healthy individuals of Flemish ancestry via the Red Cross (Leuven, Belgium) and DNA from these individuals was genotyped for rs7993418. We compared sVEGFR-1 plasma levels from 30 and 28 randomly selected TT and TC carriers against each of the 11 CC (mutant) carriers via the Human Soluble VEGF R1/Flt-1 Immunoassay (R&D systems, catalog # DVR100B). We observed that CC carriers have an 18% increased median VEGFR-1 expression compared to TT and TC carriers (P=0.006). One-way ANOVA was used to evaluate the effect of rs7993418 on sVEGFR-1 expression; a two-sided P value <0.05 was considered statistically significant. We replicated this association in an independent cohort of plasma samples from breast cancer patients (collected at the Leuven Multidisciplinary Breast Center). Briefly, DNA from 263 patients was genotyped for rs7993418 and sVEGFR-1 plasma levels from 23 and 27 randomly selected TT (wildtype) and TC carriers was compared against each of the 9 detected CC (mutant) carriers. A similar increase in sVEGFR-1 expression (19%) was noticed in CC carriers versus TT and TC carriers (P=0.014). One-way ANOVA was used to evaluate the effect of rs7993418 on sVEGFR-1 expression; a two-sided P value <0.05 was considered statistically significant.

3. VEGFR-1 Expression in HUVECs Stratified for rs7993418 Genotypes

Finally, by comparing HUVECs that carry rs7993418 TT, TC and CC genotypes, we could not identify any differences for tmVEGFR-1 (P=0.50) and sVEGFR-1 (P=0.91) mRNA expression levels. However, similar to the in vitro translation experiments, these HUVECs showed slightly increased tmVEGFR-1 protein expression levels for CC carriers versus TT or TC carriers (23% increase; P=0.049). Similar effects between CC versus TC or TT carriers were observed for sVEGFR-1 (39% increase; P=0.044).

### 4. ERK1/2 Activation Upon PlGF Stimulation

The above findings indicate that rs7993418, by enhancing mRNA translation efficacy, increases expression of tmVEGFR-1 and sVEGFR-1. Furthermore, as expected by the increase in VEGFR-1 expression, HUVEC cultures homozygous for the C-allele exhibited increased downstream VEGFR-1 signaling upon activation with the selective VEGFR-1 ligand, PlGF.

This is shown by increased levels of phospho-ERK1 and phospho-ERK2 in CC versus TT rs7993418 carriers (2.0 versus 1.6 fold induction for phospho-ERK1 and 2.1 versus 1.4 fold induction for phospho-ERK2; P=0.045 and P=0.046; n=3 versus 5). ERK1 and ERK2 phosphorylation was measured using the Phospho-MAPK array kit (R&D systems). Phosphorylated proteins were detected using the Pierce ECL chemiluminescent substrate (Thermo Scientific) and blots were developed using Scientific imaging film (Kodak). Blots were scanned and intensities were quantified using the ImageJ 1.43 software. Intensities were background corrected and scaled relative to the internal positive control of the Phospho-MAPK array kit. Experiments were performed twice and average values of both experiments are shown. Since the Human Phospho-MAPK array kit does not correct for the total amount of ERK1 or 2, we measured total ERK1/2 concentrations using SureFire technology (Perkin Elmer). Total ERK1/2 levels were similar for TT and CC carriers under unstimulated and stimulated conditions (P=0.2 and 0.34, respectively).

### Association of the VEGFR-1 Locus Replicates in AVOREN

Finally, in an attempt to replicate the association of the VEGFR-1 locus with bevacizumab treatment outcome, we investigated a second phase III clinical study involving metastatic renal cell carcinoma patients (AVOREN). Blood samples from AVOREN were available from 110 out of 649 patients (16.9%), 59 of which received bevacizumab (Table 4).

TABLE 4
AVOREN patient demographics and clinical characteristics at baseline
Demographics and characteristics are given for the full AVOREN trial cohort and
for the subgroup available for genetic biomarker analysis. Bev denotes bevacizumab; CI
confidence interval, IFN Interferon alfa-2a, mo months.

| | AVOREN population | | | | Biomarker subgroup | | | |
|---|---|---|---|---|---|---|---|---|
| | IFN | | Bev + IFN | | IFN | | Bev + IFN | |
| Characteristic | (N = 322) | | (N = 327) | | (N = 51) | | (N = 59) | |
| Sex - no. (%) | | | | | | | | |
| Female | 87 | (27) | 105 | (32) | 13 | (25) | 29 | (29) |
| Male | 235 | (73) | 222 | (68) | 38 | (75) | 48 | (71) |
| Age category - no. (%) | | | | | | | | |
| <65 yr | 204 | (63) | 206 | (63) | 30 | (59) | 37 | (63) |
| ≧65 yr | 118 | (37) | 121 | (37) | 21 | (41) | 22 | (37) |
| Smoking status - no. (%) | | | | | | | | |
| Current smoker | 43 | (13) | 45 | (14) | 9 | (18) | 6 | (10) |
| Past smoker | 129 | (40) | 126 | (39) | 20 | (39) | 23 | (39) |
| Never smoker | 148 | (46) | 154 | (47) | 22 | (43) | 30 | (51) |
| Unknown | 2 | (<1) | 2 | (<1) | 0 | (0) | 0 | (0) |
| Progression-free survival | | | | | | | | |
| Patients with event - no. (%) | 298 | (92.5) | 301 | (92.0) | 42 | (82.4) | 56 | (94.9) |
| Patients without event - no. (%) | 24 | (7.5) | 26 | (8.0) | 9 | (17.6) | 3 | (5.1) |
| Median time to event - | 5.5 | (4.2-5.7) | 10.2 | (7.7-11.1) | 8.7 | (7.2-14.2) | 15.5 | (13.5-18.4) |
| mo (95% CI) | | | | | | | | |
| Hazard ratio (95% CI) | 0.75 (0.64-0.88) | | | | 0.93 (0.62-1.40) | | | |
| Overall survival | | | | | | | | |
| Patients with event - no. (%) | 224 | (69.6) | 220 | (67.3) | 28 | (54.9) | 30 | (50.8) |
| Patients without event - no. (%) | 98 | (30.4) | 107 | (32.7) | 23 | (45.1) | 29 | (49.2) |
| Median time to event - | 21.3 | (18.4-24.5) | 23.3 | (20.4-27.0) | 37.2 | (28.2-39.7) | 34.9 | (30.0-.) |
| mo (95% CI) | | | | | | | | |

| | | |
|---|---|---|
| Hazard ratio (95% CI) | 0.91 (0.76-1.10) | 0.93 (0.55-1.55) |

A similar SNP analysis as for the AVITA trial, as described above, was performed on the genetic samples from patients in the AVOREN trial. Since AVOREN patients receiving bevacizumab switched to heterogeneous second-line therapies upon disease progression, we only assessed correlation to PFS. Although the genetic biomarker subgroup was characterized by a longer PFS than the full patient cohort, rs7993418 correlated with PFS in the bevacizumab (per-allele HR=1.8, P=0.033, Table 5), but not in the placebo arm (per-allele HR=0.8, P=0.49).

TABLE 5
Kaplan-Meier estimates of PFS
in bevacizumab and placebo treated groups in AVOREN,
according to rs7993418, rs9554316 and rs9513070 genotype.

Progression-Free Survival
Median time to event - mo

| | IFN | Bev + IFN |
|---|---|---|
| Genotype | (N = 51) | (N = 59) |
| rs7993418 or rs9554316* | | |
| TT | 7.95 (N = 26) | 16.66 (N = 36) |
| TC | 13.37 (N = 17) | 10.15 (N = 18) |
| CC | 8.11 (N = 2) | 14.52 (N = 1) |
| Hazard ratio (95% CI) | 0.83 (0.47-1.44) | 1.81 (1.08-3.05) |
| P value | 0.49 | 0.033 |

Bev denotes bevacizumab; CI confidence interval, IFN Interferon alfa-2a, mo months.

* Rs7993418 and rs9554316 were synonymous to each other and the analysis in Table 5 was conducted on rs9554316.

In Table 5, the CC genotype of rs7993418 involves only one patient; therefore, no conclusion can be drawn about the median survival of CC carries. Nevertheless, the combined genotypic effect by both TC and CC carriers of rs7993418 in AVOREN is statistically significant (P=0.033). The results shown in Table 5 together with the above-described functional characterization of rs7993418 support the findings of the present invention that the C allele adversely affects the survival in bevacizumab-treated patients by increasing expression of VEGFR-1, which could amplify the well-recognized phenomenon of compensatory angiogenesis driven by the PlGF ligand.

Overall, this indicates that the VEGFR-1 locus may also be predictive for bevacizumab treatment outcome in renal cell carcinoma patients.

A genetic locus in the VEGFR-1 TK domain that is associated with PFS and OS in metastatic pancreatic cancer patients (AVITA) has been identified in the present invention and replicated with PFS in renal cell carcinoma patients (AVOREN). Importantly, this association was specific for patients receiving bevacizumab as no significant effects were observed in placebo-treated patients. We also validated this locus at the functional level by demonstrating that rs7993418 enhances VEGFR-1 mRNA translation efficacy, leading to increased expression of VEGFR-1 protein.

Concerning how increased VEGFR-1 expression could contribute to reduced bevacizumab treatment outcome, it is well known that activation of VEGFR-1 triggers angiogenesis, either directly by transmitting intracellular signals or indirectly by transphosphorylation of VEGFR-2, resulting in increased VEGFR-2-driven angiogenesis. Interestingly, tumors that overexpress the VEGFR-1 selective ligand, PlGF, grow less rapidly in mice lacking the VEGFR-1 TK domain as a result of reduced vascularization of these tumors. Since PlGF levels are also increased in patients treated with bevacizumab, a genetic locus that amplifies downstream signaling of VEGFR-1 could render the vasculature more dependent on PlGF and cause resistance to anti-VEGF treatment. Similarly, increased sVEGFR-1 levels can sequester tumor-derived VEGF, thereby reducing its pro-angiogenic effects transduced via VEGFR-2 and limiting the benefits of VEGF neutralization through bevacizumab. Indeed, Mazzone et al. have shown that high tmVEGFR-1 and sVEGFR-1 expressing endothelial cells contribute to tumor vasculature normalization, in part because these cells are less responsive to the mitogenic and migratory activity of VEGF (Mazzone M, Dettori D, Leite de Oliveira R, et al. Heterozygous deficiency of PHD2 restores tumor oxygenation and inhibits metastasis via endothelial normalization. Cell 2009; 136:839-51). Remarkably, rectal cancer patients with increased plasma sVEGFR-1 expression before and during treatment have a reduced benefit from bevacizumab, thereby underscoring the observations of the present invention about the potential value of VEGFR-1 as a biomarker of bevacizumab treatment (Duda D G, et al. Plasma soluble VEGFR-1 is a potential dual biomarker of response and toxicity for bevacizumab with chemoradiation in locally advanced rectal cancer. Oncologist. 2010; 15 (6):577-83)

At first glance, it may seem surprising that a synonymous SNP affects VEGFR-1 expression without changing the amino acid sequence. However, synonymous mutations have previously been reported to affect protein expression and have already been implicated in >40 diseases. One potential mechanism whereby synonymous SNPs may affect protein expression is through codon bias. In particular, the rs7993418 variant may affect codon usage of the tyrosine located on position 1213 in the TK domain of VEGFR-1. This domain is characterized by a strong bias towards TAC codons, i.e. 16 TAC codons versus 5 TAT codons, which both code for a tyrosine. Such codon bias is also present in highly expressed genes across various species, in which it represents a mechanism to promote efficient translation of highly expressed genes. More efficient VEGFR-1 translation induced by the TAC codon can be achieved through various mechanisms, including i) the more favorable interaction of the TAC codon with its tRNA anticodon due to the stronger G-C hydrogen bond interaction at the third codon position (Grosjean H, Fiers W. Preferential codon usage in prokaryotic genes: the optimal codon-anticodon interaction energy and the selective codon usage in efficiently expressed genes. Gene 1982; 18:199-209), ii) increased availability of tRNAs for the TAC codon (the TAT tRNA is encoded by only a single gene, whereas 14 tRNA genes exist for TAC) (Juhling F, Morl M, Hartmann R K, Sprinzl M, Stadler P F, Putz J. tRNAdb 2009: compilation of tRNA sequences and tRNA genes. Nucleic Acids Res 2009; 37:D159-62), and iii) the effect of ‘tRNA recycling’ by ribosomes, which favors re-use of the most frequently-used codon to improve translation efficacy (Cannarozzi G, Schraudolph N N, Faty M, et al. A role for codon order in translation dynamics. Cell; 141:355-67). All together, these mechanisms support the notion that codon bias mediates the effect of rs7993418 on VEGFR-1 expression and its association with treatment outcome of bevacizumab.

What is claimed is:

1. An in vitro method of determining whether a patient suffering from cancer or physiological or pathological angiogenic abnormalities is suitably treated by a therapy with an angiogenesis inhibitor comprising bevacizumab or an antibody that binds essentially the same epitope on VEGF as bevacizumab, said method comprising:
(a) determining in a sample derived from a patient suffering from cancer or physiological or pathological angiogenic abnormalities the genotype at the synonymous T/C SNP located in exon 28 of VEGFR-1 corresponding respectively to TAT codon and TAC codon for tyrosine at position 1213, and
(b) identifying said patient as more or less suitably treated by a therapy with an angiogenesis inhibitor comprising bevacizumab or an antibody that binds essentially the same epitope on VEGF as bevacizumab based on said genotype, wherein the presence of each T allele at said SNP indicates an increased likelihood that the patient is more suitably treated, or the presence of each C allele at said SNP indicates an increased likelihood that the patient is less suitably treated.

2. The method of claim 1, wherein whether a patient is suitably treated by a therapy with an angiogenesis inhibitor is determined in termes of whether progression-free survival or overall survival is improved.

3. The method of any one of claims 1 to 2, wherein the method further comprises treating the patient by the therapy with an angiogenesis inhibitor.

4. The method of any one of claims 1 to 3, wherein the angiogenesis inhibitor is administered as a co-treatment with a chemotherapeutic agent or chemotherapy regimen.

5. The method of any one of claims 1 to 4, wherein the angiogenesis inhibitor is administered with one or more agents selected from the group consisting of taxanes, interferon alpha, 5-fluorouracil, capecitabine, leucovorin, gemcitabine, erlotinib and platinum-based chemotherapeutic agents.

6. The method of any one of claims 1 to 5, wherein the cancer is pancreatic cancer, renal cell cancer, colorectal cancer, breast cancer or lung cancer.

7. A pharmaceutical composition comprising an angiogenesis inhibitor comprising bevacizumab or an antibody that binds essentially the same epitope on VEGF as bevacizumab for the treatment of a patient suffering from cancer or physiological or pathological angiogenic abnormalities, wherein the patient has been identified as more suitably treated with the angiogenesis inhibitor in accordance with the method of any one of claims 1 to 6.

8. A kit for carrying out the method of any one of claims 1 to 6, comprising oligonucleotides capable of determining the genotype at the synonymous T/C SNP located in exon 28 of VEGFR-1 corresponding respectively to TAT codon and TAC codon for tyrosine at position 1213.

9. A method for improving the treatment effect of a chemotherapeutic agent or chemotherapy regimen of a patient suffering from cancer or physiological or pathological angiogenic abnormalities by adding an angiogenesis inhibitor comprising bevacizumab or an antibody that binds essentially the same epitope on VEGF as bevacizumab, said method comprising:
(a) determining in a sample derived from a patient suffering from cancer or physiological or pathological angiogenic abnormalities the genotype at the synonymous T/C SNP located in exon 28 of VEGFR-1 corresponding respectively to TAT codon and TAC codon for tyrosine at position 1213;
(b) identifying said patient as more suitably treated by the addition of an angiogenesis inhibitor comprising bevacizumab or an antibody that binds essentially the same epitope on VEGF as bevacizumab based on said genotype, wherein the presence of each T allele at said SNP indicates an increased likelihood that the patient is more suitably treated; and
(c) administering said angiogenesis inhibitor in combination with a chemotherapeutic agent or chemotherapy regimen to the patient identified as more suitably treated in accordance with (b).

10. The method of claim 9, wherein whether a patient is suitably treated by a therapy with an angiogenesis inhibitor is determined in terms of whether progression-free survival or overall survival is improved.

11. The method of any one of claims 9 to 10, wherein the angiogenesis inhibitor is administered with one or more agents selected from the group consisting of taxanes, interferon alpha, 5-fluorouracil, capecitabine, leucovorin, gemcitabine, erlotinib and platinum-based chemotherapeutic agents.

12. The method of any one of claims 9 to 11, wherein the cancer is pancreatic cancer, renal cell cancer, colorectal cancer, breast cancer or lung cancer.


SEQUENCE LISTING (1 sequence)

SEQ ID NO: 1 - DNA, 52 bp, Homo sapiens
aggctcatgaacttgaaagcatttacrtatctaatgaagaaacagaaagaat